(19) United States
(12) Patent Application Publication
(10) Pub. No.: US 2017/0100115 A1
(43) Pub. Date: Apr. 13, 2017

(54) LAPAROSCOPIC SPRAY APPLICATOR AND ADAPTER

(71) Applicant: MEDMIX SYSTEMS AG, Rotkreuz (CH)

(72) Inventors: Beat MATHYS, Muri (CH); Andy GRETER, Minusio (CH)

(73) Assignee: MEDMIX SYSTEMS AG, Rotkreuz (CH)

(21) Appl. No.: 15/312,001

(22) PCT Filed: Apr. 22, 2015
(86) PCT No.: PCT/EP2015/058700

§ 371 Date: Nov. 17, 2016

(30) Foreign Application Priority Data
May 22, 2014 (CH) 782/14

(51) Int. Cl. A61B 17/00

(52) U.S. Cl. CPC A61B 17/00491; A61B 2017/00495; A61B 2017/00522; A61B 2017/00477

(57) ABSTRACT

An applicator for spraying at least two components in the interior of the body of a patient uses a compressed gas, the applicator being suitable in particular for laparoscopic applications. The applicator has a connecting piece for connection of the applicator to two reservoirs for the components and for delivery of a compressed gas. A multi-lumen tube extends from the connecting piece to a spray head. A flexible wire is fixed in a first lumen which extends off-center. The applicator has at least one adapter having two tube connectors which are introduced into two further lumina. These two further lumina likewise extend off-center and are offset in the circumferential direction from the lumen which receives the wire. A further lumen can extend centrally and can receive a second wire. Identical adapters can be provided at both ends of the multi-lumen tube.

### TECHNICAL FIELD

The present invention relates to an applicator for spraying at least two components in the interior of the body of a patient by using a compressed gas, and to an adapter for use in such an applicator.

### PRIOR ART

Minimally invasive surgical procedures have become a permanent part of daily medical routine. In such procedures, surgical instruments are introduced into the body through small incisions. Laparoscopic procedures are one important class of minimally invasive procedures. In these procedures, surgical instruments are introduced into the abdominal cavity of the patient through the abdominal wall in order to perform operations on organs that are accessible from the abdominal cavity.

One particular challenge associated with minimally invasive procedures is that of halting bleeding that can occur in the interior of the body during the surgical procedure. In particular, it is very difficult to close incisions and other lesions in the interior of the body by means of a surgical suture, which is a widespread practice in traditional surgical procedures.

In recent years, the practice of using tissue adhesives in order to halt bleeding has become increasingly established. Such tissue adhesives usually consists of two or more components which react with one another very rapidly and, therefore, must be kept separate from one another until shortly before their application. One known example is fibrin adhesives which contain the components thrombin and fibrinogen. As soon as the components of such a tissue adhesive are mixed, they coagulate very rapidly and then can no longer be applied.

Tissue adhesives can be applied by spraying with the aid of a spray nozzle. In this case, the components of the tissue adhesive should be mixed with one another preferably only directly before the spray nozzle, in the spray nozzle itself, or even only after having emerged from the spray nozzle.

Another important objective associated with minimally invasive procedures is to prevent areas of tissue in the interior of the body from growing together in an uncontrolled manner (adhesion). Adhesion is one of the most frequent complications occurring after surgery in the abdominal region. A way to effectively prevent adhesion is therefore essential, especially also in the case of laparoscopic procedures. So-called adhesion barriers can be utilized in order to prevent adhesion. These are (frequently gel-like) substances which are applied onto affected areas of tissue in order to prevent adhesion by another tissue. Adhesion barriers, similar to tissue adhesives, can be advantageously applied by being sprayed on. Adhesion barriers can also be formed from two components which are preferably mixed only just before or during application. The objectives associated with the application of adhesion barriers are therefore similar to those associated with the application of tissue adhesives.

Different applicators for spraying two-component systems in a laparoscopic application have become known from the prior art.

One first example of a spray applicator for laparoscopic applications is disclosed in U.S. Pat. No. 7,682,336. In this case, two rigid tubes are enclosed by a rigid, cylindrical sleeve. The two components of a tissue adhesive are guided through the tubes to an exchangeable spray head, while a compressed gas is guided through the intermediate space between the tubes and the sleeve to the spray head. The components are mixed in the spray head and are sprayed with the aid of the compressed gas. This applicator has the disadvantage that it is entirely rigid. As a result, it is only possible to spray in the direction of the longitudinal axis of the sleeve. Sites in the interior of the body that are difficult to access may therefore not be reachable by using this applicator.

A further example of a spray applicator for laparoscopic applications is disclosed in U.S. Pat. No. 8,303,531. The applicator includes a sleeve in which a flexible multi-lumen tube is located. The multi-lumen tube extends beyond the sleeve, in the distal direction. The multi-lumen tube includes a central lumen and four decentral lumina which are distributed around the circumference of said central lumen. A flexible wire is inserted into the central lumen. Connectors are provided at the proximal end of the sleeve in order to connect the applicator to a multi-tube syringe and a compressed gas source. From there, the components of the spray and the compressed gas are guided through the lumina of the multi-lumen tube to a spray head at the distal end of the multi-lumen tube. In the spray head, the components are mixed with the compressed gas and are sprayed. The distal area of the multi-lumen tube, which extends beyond the sleeve, can be bent. As a result, it is possible to also spray in a direction, using this applicator, which does not extend along the longitudinal axis of the sleeve. It is difficult, however, to precisely adjust the orientation of the bent area with respect to a rotation about the longitudinal axis of the sleeve (i.e., the azimuth angle of the bent area). It has also been shown that the bent area can abruptly change its azimuthal orientation when torsional forces act on the distal end of the applicator. The document also does not describe in detail how the connector housing and the spray head are connected to the flexible multi-lumen tube. It can be difficult to establish a reliable connection at these points.

### SUMMARY OF THE INVENTION

In a first aspect, the invention provides an applicator for spraying at least two components in the interior of the body of a patient by using a compressed gas, wherein the applicator can be easily produced despite its compact dimensions and provides for reliable operation.

The proposed applicator comprises:
a connecting piece for connecting the applicator to reservoirs for the components and for feeding a compressed gas to the applicator;
a multi-lumen tube which defines a central longitudinal axis and has a proximal end, which is connected to the connecting piece, and a distal end;
a spray head which is located at the distal end of the multi-lumen tube, and
at least a first flexible wire which is arranged in a first lumen of the multi-lumen tube.

In order to provide for simple assembly, the applicator comprises at least one adapter which is located at the proximal or distal end of the multi-lumen tube in order to connect the multi-lumen tube to the connecting piece or to the spray head. The adapter has two tube connectors which are introduced into a second and a third lumen of the multi-lumen tube. The second and the third lumina are used for guiding fluid. These are preferably the lumina that guide the components to be sprayed. Each of these lumina preferably communicates with one respective reservoir connector on the connecting piece. These two lumina extend off-center with respect to the longitudinal axis. They also preferably extend diametrically opposite one another with respect to the longitudinal axis. The first lumen, in which the first flexible wire is located, likewise extends, according to the invention, off-center with respect to the longitudinal axis. Said first lumen also extends offset from the second and third lumen in the circumferential direction. Due to this arrangement of the wire-guiding first lumen, which is off-center and is offset in the circumferential direction, it is possible to provide a sufficiently large cross-section for the component-guiding lumina without excessively increasing the outer diameter of the multi-lumen tube.

The second and the third lumina preferably each have a larger cross-section than the first lumen. When the second and the third lumina extend diametrically opposite one another, the first lumen preferably extends offset by approximately 90° (e.g., by 80°-100°) from the second and the third lumina about the longitudinal axis. In other words: In a diametrically opposed arrangement, the second and the third lumina jointly define a first plane which contains the central axes of both of these lumina and the central longitudinal axis of the multi-lumen tube. The central axis of the first lumen then preferably extends in a second plane which contains the central longitudinal axis of the multi-lumen tube and extends perpendicular to the first plane.

In addition, further lumina can be provided, in particular a fourth lumen for the guidance of compressed gas, which communicates with a compressed gas connector on the connecting piece. The lumen for the guidance of compressed gas preferably extends diametrically opposite the first lumen which accommodates the wire.

The invention solves a dilemma between the requirement for simple production and the requirement for compact dimensions. The adapter results in simpler production, and a reliable guidance of fluid is achieved. On the other hand, the cross-section of such lumina of the multi-lumen tube, which are provided for the guidance of fluid, must be sufficiently large to accommodate the tube connectors of the adapter. This stands in contrast to the requirement that the multi-lumen tube have the smallest outer diameter possible. This outer diameter affects the size of the opening that must be created in the body in order to introduce the applicator into the interior of the body. Given that the wire-guiding lumen is situated so as to be off-center, place is created for an enlarged cross-section of the component-guiding lumina without enlarging the outer diameter of the multi-lumen tube.

Preferably, the applicator comprises two adapters which are identically designed and, preferably, are even completely identical. In this case, one of the two adapters is arranged at the proximal end of the multi-lumen tube in order to connect the multi-lumen tube to the connecting piece, and the other adapter is arranged at the distal end of the multi-lumen tube in order to connect the multi-lumen tube to the spray head. Due to this design of the applicator, assembly and stockkeeping are additionally simplified, since, on the one hand, work can be performed with adapters at both ends and, on the other hand, only one single type of adapter is required.

Preferably at least sections of the first flexible wire are fixed in the multi-lumen tube (i.e., are secured against any movements such as rotation or displacement relative to the multi-lumen tube), in particular being bonded to the multi-lumen tube or cast into the multi-lumen tube, or extruded therein. If the wire is bonded with the multi-lumen tube, a suitable adhesive between the wire and the outer wall of the first lumen can be located at least along one or more sections of the wire. The adhesive is preferably a UV-cured adhesive, and the multi-lumen tube in this case is preferably permeable to UV light. The wire is preferably fixed at least at its ends, and particularly preferably essentially along its entire length in the multi-lumen tube. Due to the fixing, it is ensured that the bent area of the multi-lumen tube retains its orientation relative to the connecting piece even when torsional forces act on the distal area of the applicator.

Such a fixation of the wire in the lumen is advantageous regardless of whether the applicator comprises an adapter of the above-described type and regardless of how the lumina of the multi-lumen tube are arranged.

In some embodiments, the applicator can comprise a second flexible wire which is arranged in a further lumen of the multi-lumen tube. The second wire is preferably likewise fixed in the multi-lumen tube, at least in sections, preferably at least at its ends, particularly preferably along its entire length, as described above. The further lumen can extend centrally along the longitudinal axis. In this case, although this lumen requires additional space in the center between the lumina for the guidance of the fluid, other advantages are achieved as a result, which offset this disadvantage to a certain extent. In this way, due to the presence of two parallel wires, it is achieved in particular that the bending moment required for bending the multi-lumen tube differs in different directions (i.e., it is anisotropic with respect to the azimuthal direction). The bending moment is greater for bending motions that take place in a plane that extends through both wires, than for motions in a plane perpendicular thereto. This can be, e.g., deliberately taken advantage of in order to specifically bend the multi-lumen tube, before the application, only in the latter plane. A relatively small bending torque, i.e., a relatively small force, is required therefor, which can be easily applied by the user. Unintentional lateral motions of the bent area in the plane that extends through both wires are then minimized during the application due to the much greater bending moment that is required therefor.

The adapter can be designed as follows, in particular: Said adapter comprises a base body which defines a first side and a second side with respect to a longitudinal direction. The aforementioned first and second tube connectors protrude from the base body, on the first side (preferably essentially parallel to the longitudinal direction). The adapter also comprises a first and a second inlet/outlet connector. Said connectors protrude from the base body, on the second side (preferably essentially parallel to the longitudinal direction). Each one is designed to be inserted into an opening of a further element. A first component channel extends through the base body and connects the first tube connector to the first inlet/outlet connector. Correspondingly, a second component channel also extends through the base body and connects the second tube connector to the second inlet/outlet connector. In addition, the adapter comprises at least one compressed gas channel which connects the first side and the second side of the base body. Said adapter is designed for communicating with a lumen, which extends off-center, for the guidance of compressed gas.

When such an adapter is located at the distal end of the multi-lumen tube in order to connect the multi-lumen tube to the spray head, the applicator can be designed in this area as follows: The spray head can comprise a spray head insert and a spray head cap. The spray head insert then has a first component inlet, into which the first inlet/outlet connector of the adapter is inserted, and a second component inlet, into which the second inlet/outlet connector of the adapter is inserted. The spray head insert can also comprise at least one lateral recess which is designed in such a way that the spray head insert, together with the spray head cap and the adapter, delimits a hollow space in the area of the lateral recess, which the compressed gas enters after having emerged from the compressed gas channel of the adapter and before it emerges from the spray head. In this way, a clean guidance of the components through the spray head and a uniform distribution of compressed gas are achieved.

The outer wall of the spray head cap can radially surround the multi-lumen tube at its distal end. It is then advantageous when the spray head cap is bonded, in the area of the outer wall, to the multi-lumen tube by means of an adhesive. The adhesive is preferably UV-cured. At least the outer wall of the spray head cap is then preferably permeable to UV light.

The guidance of the components and the gas at the spray head tip preferably takes place as follows: The spray head insert comprises a first component channel which is connected to the first component inlet and forms a first component outlet at the spray head tip. Correspondingly, the spray head insert comprises a second component channel which is connected to the second component inlet and forms a second component outlet at the spray head tip. Multiple gas outlet channels are formed between the spray head insert and the spray head cap, which lead out of the spray head in an area adjacent to the component outlets.

When such an adapter of the aforementioned type is located at the proximal end of the multi-lumen tube in order to connect the connecting piece to the multi-lumen tube, the applicator can be designed in this area as follows: The connecting piece comprises a first and a second reservoir connector for connection to the reservoirs, and a compressed gas connector for feeding the compressed gas. The adapter then connects the reservoir connectors to the multi-lumen tube. In this case, the connecting piece can have a tubular holding area having an open, distal end and a proximal end. The compressed gas connector leads into the interior of the tubular holding area in this case. The multi-lumen tube is then slid from the open distal end into the tubular holding area. The connecting piece has, at the proximal end of the holding area, a first component feed opening which communicates with the first reservoir connector and into which the first inlet/outlet connector of the adapter is inserted. Correspondingly, the connecting piece also has, at the proximal end of the holding area, a second component feed opening which communicates with the second reservoir connector and into which the second inlet/outlet connector of the adapter is inserted. The multi-lumen tube has, in the area of the compressed gas connector, a lateral opening which connects the compressed gas connector to one of the lumina of the multi-lumen tube. In this manner, a simple and clean feed of the components to the multi-lumen tube and a reliable compressed gas supply are achieved.

It is advantageous when the multi-lumen tube and the adapter are fixed in the holding area by means of an adhesive, in an area which is located proximal to the lateral opening. Additionally, it is advantageous when the multi-lumen tube is fixed in the holding area in a gas-tight manner in an area located distal to the lateral opening, preferably likewise by means of an adhesive. In this way, compressed gas is prevented from escaping from the holding area to the outside or is prevented from entering the lumina provided for the components.

The applicator can also comprise a sleeve which extends from the connecting piece in the distal direction and which radially encloses at least a portion of the multi-lumen tube, as is known per se from the prior art. In this case, it is advantageous when the sleeve is likewise fixed by an adhesive in the holding area, in an area located distal to the lateral opening.

In order to feed the adhesive to the area located proximal to the lateral opening, the connecting piece can comprise an adhesive channel which is accessible from the outside and leads into the aforementioned area. The adhesive, in turn, is preferably UV-cured and the connecting piece is then permeable to UV light advantageously at least in an area in which the adhesive is present.

In a third aspect, the present invention provides an adapter which allows for simple production and a reliable design of a spray applicator for laparoscopic applications.

The adapter according to the invention is intended for use in an applicator for spraying at least two components in the interior of the body of a patient by using a compressed gas, in particular in an applicator of the above-described type. It comprises:
a base body which defines a first side and a second side with respect to a longitudinal direction;
a first and a second tube connector, wherein each of the tube connectors protrudes from the base body, on the first side (preferably essentially parallel to the longitudinal direction), and is designed for insertion into a lumen of a multi-lumen tube,
a first and a second inlet/outlet connector, wherein each of the inlet/outlet connectors protrudes from the base body, on the second side (preferably essentially parallel to the longitudinal direction) and is designed for insertion into an opening of a further element,
a first component channel which extends through the base body and connects the first tube connector to the first inlet/outlet connector,
a second component channel which extends through the base body and connects the second tube connector to the second inlet/outlet connector, and
at least one compressed gas channel which connects the first side and the second side of the base body.

The base body can have a lateral recess on the first side, and the compressed gas channel can then terminate on the first side of the base body in the area of the lateral recess.

In preferred embodiments, the adapter comprises two compressed gas channels. The base body then has two lateral recesses which are situated diametrically opposite one another, and both compressed gas channels terminate on the first side of the base body in the area of the lateral recesses.

At least one retaining structure can be formed on both the first and the second tube connectors, which prevents the particular tube connector from being pulled back out of the multi-lumen tube. A sealing bead for sealing between the relevant inlet/outlet connector and the further element can be formed on both the first and the second inlet/outlet connector.

### BRIEF DESCRIPTION OF THE DRAWINGS

Preferred embodiments of the invention are described in the following with reference to the drawings which are intended merely for the purpose of explanation and are not intended to be interpreted to be limiting. In the drawings:

FIG. 1 shows a discharge device comprising a laparoscopic spray applicator according to a first embodiment;

FIG. 2 shows a detailed view of the spray applicator in an exploded representation;

FIG. 3 shows a cross-section of the multi-lumen tube of the spray applicator;

FIG. 4 shows an enlarged view of the area A in FIG. 2;

FIG. 5 shows an enlarged view of the area B in FIG. 2;

FIG. 6 shows a first perspective view of an adapter of the spray applicator;

FIG. 7 shows a second perspective view of an adapter of the spray applicator;

FIG. 8 shows a front view of the spray applicator in the proximal direction;

FIG. 9 shows a longitudinal section of the spray applicator in the plane A-A from FIG. 8;

FIG. 10 shows a longitudinal section of the spray applicator in the plane B-B from FIG. 8;

FIG. 11 shows an enlarged representation of the area X from FIG. 9;

FIG. 12 shows an enlarged representation of the area Z from FIG. 10;

FIG. 13 shows a perspective view of the spray insert of the spray applicator;

FIG. 14 shows an enlarged representation of the area Y from FIG. 9 (distal end area);

FIG. 15 shows an enlarged representation of the area W from FIG. 10;

FIG. 16 shows a longitudinal section of the distal end area in a cutting plane which has been rotated through 45° with respect to the plane A-A about the longitudinal axis in the direction of the plane B-B;

FIG. 17 shows a cross-section of the multi-lumen tube of a spray applicator according to a second embodiment in the area of the wires extending therein; and

FIG. 18 shows a detailed view of the distal end of the spray applicator according to the second embodiment in an exploded representation.

### DESCRIPTION OF PREFERRED EMBODIMENTS

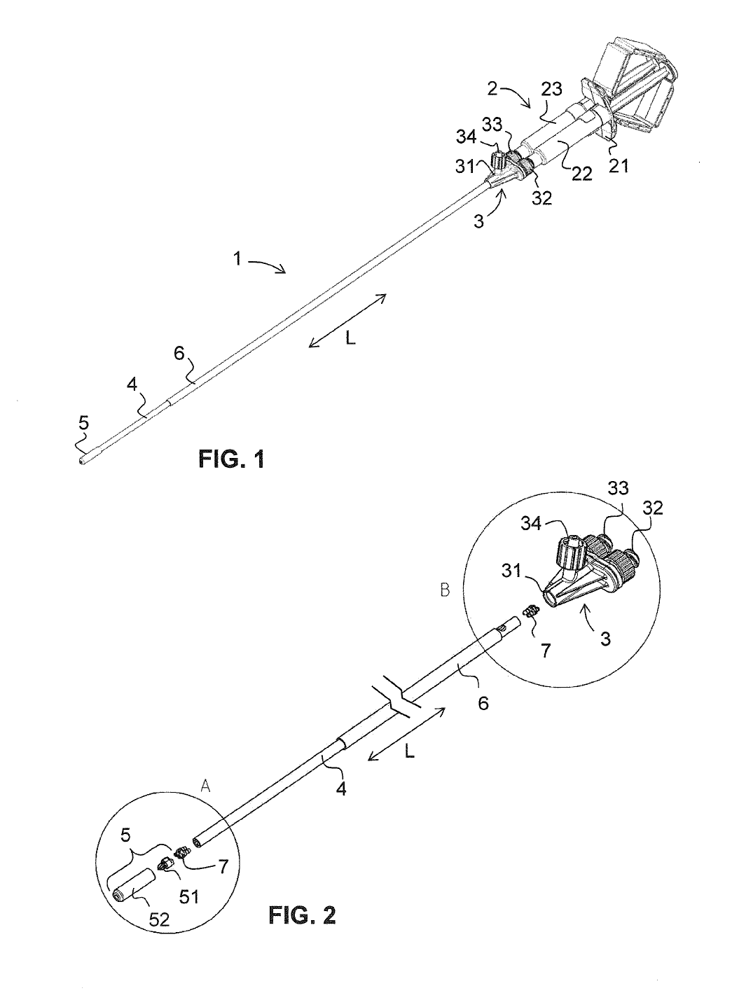

A first exemplary embodiment of a spray applicator for laparoscopic applications according to the invention is illustrated in FIGS. 1-16.

The basic design of the spray applicator 1 is clear from FIG. 1. The spray applicator 1 comprises a connecting piece 3, from which a flexible multi-lumen tube 4 having four parallel lumina extending off-center extends in the distal direction along a longitudinal direction L. A spray head 5 is mounted at the distal end of the multi-lumen tube 4. A tubular sleeve 6 (in other words, a concentric tube) surrounds a proximal area of the multi-lumen tube 4, wherein the multi-lumen tube 4 extends in the distal direction beyond the distal end of the sleeve 6 and is exposed between the distal end of the sleeve 6 and the spray head 4.

A discharge device 2 is detachably connected to the spray applicator 1. The discharge device 2 comprises a syringe holder 21 and two syringes 22, 23. Each of these syringes forms a reservoir for a component of a substance to be applied laparoscopically, e.g., a tissue adhesive or an adhesion barrier. The syringe holder 21 holds the two syringes 22, 23 parallel to one another and couples the pistons of the two syringes in such a way that they can only be advanced simultaneously in the two syringe reservoirs. With respect to the rest of the design of the discharge device 2, reference is made to U.S. Pat. No. 8,240,511 B2 (Greter et al.), the content of which is incorporated, in entirety, into the present disclosure by reference.

The connecting piece 3 comprises a main body 31, on which two reservoir connectors 32, 33 and one compressed gas connector 34 are formed. Each of the outlets of the two syringes is connected to one of the reservoir connectors 32, 33. Securing sleeves 321, 331 (see FIG. 5) secure the syringes on the connecting piece 3. With respect to the design and the function of the securing sleeves, reference is made to US 2013/0023833 A1 (Kayser), the content of which is incorporated, in entirety, into the present disclosure by reference.

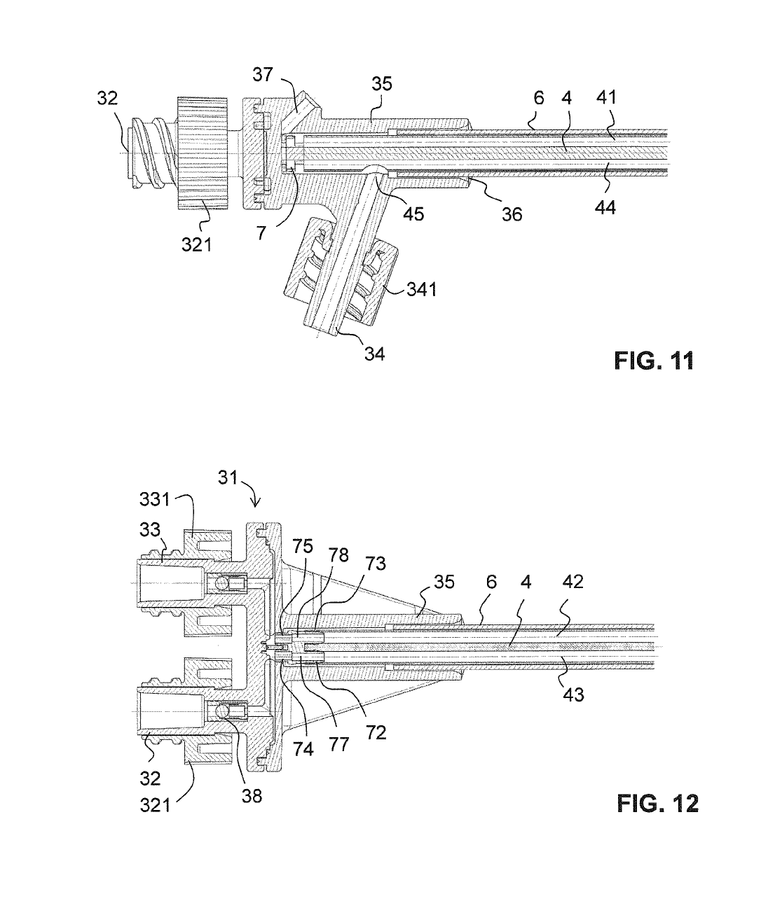

In order to spray the components out of the reservoirs, a compressed gas is fed at the compressed gas connector via a suitable compressed gas tube. A securing sleeve 341 (see FIG. 11) secures the compressed gas tube on the connecting piece 3. The compressed gas is guided through one of the lumina of the multi-lumen tube 4 to the spray head 5. As a result of pressing on the syringe pistons, the components are delivered from the syringes 22, 23 and pass through the reservoir connectors 32, 33 and through two further lumina of the multi-lumen tube 4 and arrive at the spray head 5, separate from one another. At said spray head, the components, still separated from one another, emerge from the spray head individually and are mixed and sprayed by the compressed gas which is likewise emerging at the spray head.

The design of the spray applicator 1 is illustrated in greater detail in an exploded representation in FIG. 2. The spray head 5 is formed from two separate parts, specifically a spray head insert 51 and a spray head cap 52. An adapter 7 is provided both at the proximal end of the multi-lumen tube 4 and at its distal end. The two adapters 7 are identically designed. At the proximal end of the multi-lumen tube 4, the adapter 7 is used for connecting one of the reservoir connectors 32, 33 to one lumen each of the multi-lumen tube 4. At the distal end, the adapter 7 is used for connecting the lumina of the multi-lumen tube 4 to the spray head insert 51. This is described in greater detail in the following.

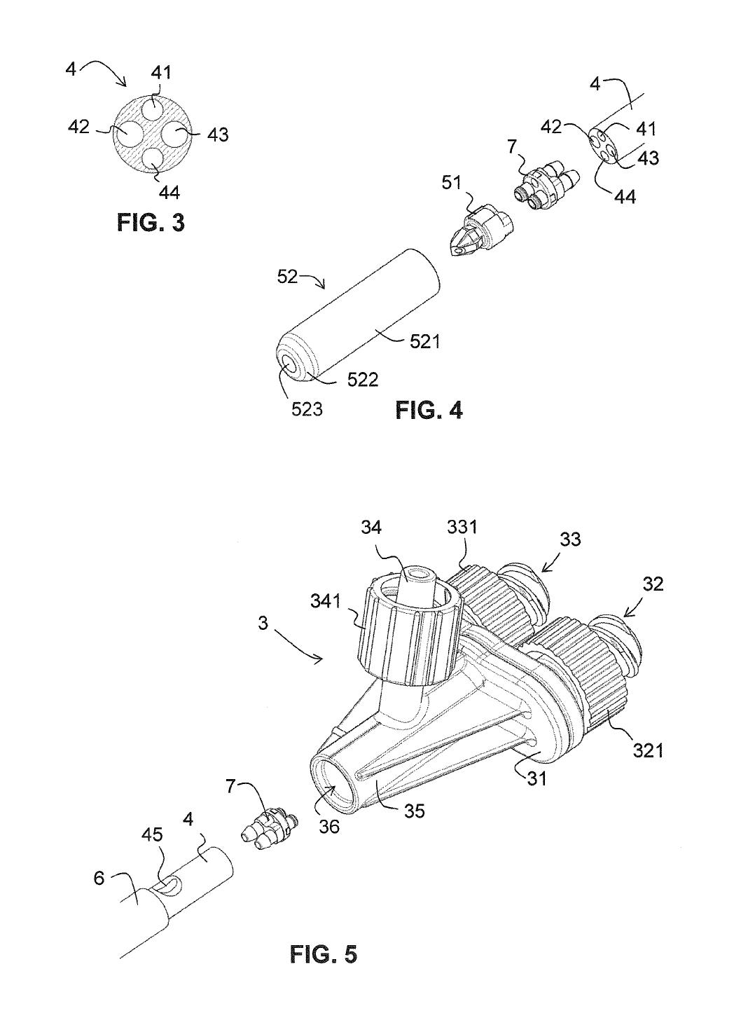

A cross-section of the multi-lumen tube 4 is represented in FIG. 3. The multi-lumen tube 4 comprises four lumina 41-44. A flexible wire is accommodated in the first lumen 41. This is described in greater detail in the following in association with FIG. 14. Two further, diametrically opposed (i.e., disposed offset from one to another by 180° about the longitudinal direction) lumina 42, 43 are provided for the guidance of the components. These lumina are disposed offset from the first lumen 41 by +/−90° about the longitudinal direction. Said lumina have a larger cross-section than the first, wire-guiding lumen 41. A further lumen 44, which is diametrically opposed to the first lumen 41, is used for guiding the compressed gas.

The connection of the lumina 41-44 of the multi-lumen tube 4 to the spray head 5 is illustrated in FIG. 4.

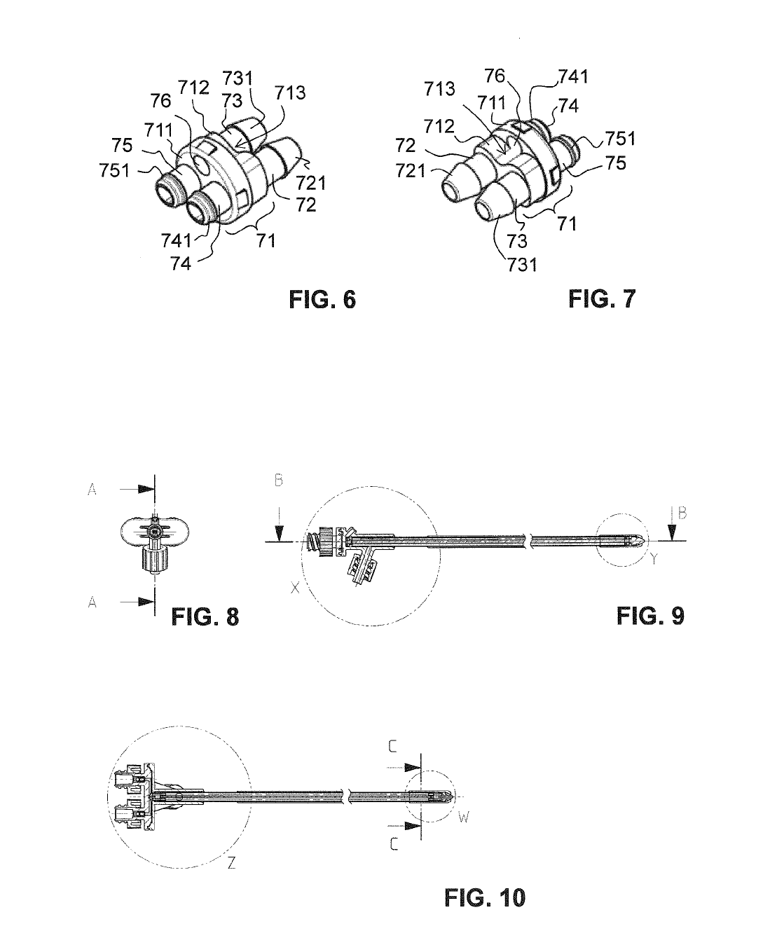

With respect thereto, reference is initially made to FIGS. 6 and 7 which show an enlarged representation of the adapter 7. The adapter 7 comprises a base body 71 which defines a first side with respect to the longitudinal direction, which faces the proximal direction (to the right) in FIG. 4, and defines a second side which faces the distal direction (to the left) in FIG. 4.

Two identical tube connectors 72, 73 protrude from the first side of the base body 71, preferably parallel to the longitudinal direction. Each of the tube connectors 72, 73 has, on it free end, a conical area 721, 731 which tapers toward the free end. A cylindrical area adjoins each conical area 721, 731 on the side facing the base body 71. The maximum diameter of the particular conical area 721, 731 is slightly greater than the diameter of the corresponding cylindrical area. A sharp edge is formed between the conical area and the cylindrical area. When the particular tube connector has been slid into the lumen of a multi-lumen tube, the sharp edge prevents the tube connector from inadvertently sliding out of the multi-lumen tube. Therefore, the sharp edge functions as a retaining structure.

Two inlet/outlet connectors 74, 75 protrude from the second side of the base body 71, preferably parallel to the longitudinal direction in this case as well. The direction in which these connectors extend is opposite the direction of the tube connectors 72, 73. The inlet/outlet connectors 74, 75 have a basic cylindrical shape. They have a sealing bead 741, 751, respectively, on their outer surface. A first component channel connects the first tube connector 72 to the first inlet/outlet connector 74, through the base body 71. Correspondingly, a second component channel connects the second tube connector 73 to the second inlet/outlet connector 75. The component channels extend radially opposite one another with respect to the longitudinal direction, in other words, are offset from one another by 180° about the longitudinal direction.

In the base body 71, two compressed gas channels 76, which are likewise radially opposed, are also present and are offset from the component channels by 90° in each case. These are relatively short. They each terminate on the first side of the base body 71 in the area of a lateral recess 713 of the base body. These channels terminate on the end face of the base body on the second side of the base body 71. The base body can be imagined to be divided into a circular disk-shaped main section 711 and a section having a reduced cross-section, which can be referred to as a spacer 712. In this case, the compressed gas channels 76 extend only through the main section 711. The spacer 712 is provided with the lateral recesses 713 and, therefore, has a smaller cross-sectional area than the main section 711. The lateral recesses 713 are disposed radially opposite one another. Overall, in the present example, a cross-sectional shape of the spacer 712 which approximately corresponds to a figure-eight therefore results. As will become apparent in the following, the spacer 712, including its recesses 713, is used for better gas guidance in the area of the spray head.

Overall, the adapter 7 is designed symmetrical with respect to a rotation by 180° about the longitudinal direction. In addition, it has mirror symmetry with respect to a horizontal mirror plane which extends through the connectors 72-75 and with respect to a mirror plane which is perpendicular thereto and contains the longitudinal direction. Due to the symmetrical design of the adapter 7, the assembly of the applicator is greatly simplified since it does not depend on an 180° rotation of the adapter about the longitudinal direction.

With respect to FIG. 4 again, it is evident that each of the tube connectors 72, 73 is inserted into one of the lumina 42, 43 of the multi-lumen tube 4 during assembly of the applicator. Each of the inlet/outlet connectors 74, 75 is also inserted into a component inlet of the spray head insert 51. This is described in greater detail in the following in association with FIG. 15. Thereupon, the spray head cap 52 is slid, in the proximal direction, onto the multi-lumen tube 4. The spray head cap 52 comprises a cylindrical outer wall 521 and a distal end wall 522. An axial spray opening 523 is centrally formed in the distal end wall. The spray head cap 52 is slid forward in the proximal direction until the distal end wall 522 impacts the spray head insert 51. The tip of the spray head insert 51 thereby protrudes distally from the spray opening 523 of the spray head cap. The spray head cap 52 is then fixed, in the area of its outer wall 521, on the multi-lumen tube 4 by an adhesive. This is preferably a UV-cured adhesive and the spray head cap 52 is therefore made from a UV-transparent plastic.

Overall, the adapter therefore establishes a connection between the lumina 42, 43 of the multi-lumen tube 4 and the spray head insert 51. In addition, compressed gas can emerge from the lumen 44 in the area of one of the recesses 713 of the adapter 7 and, from there, can continue further through the compressed gas channel 76 into the area of a corresponding recess of the spray head insert 51, from where it is conducted into gas outlet channels which are described in greater detail in the following. This is described in greater detail in the following in association with FIG. 14. As a result, the adapter 7 additionally establishes a connection from the lumen 44 to the spray head insert 51.

The connection of the connecting piece 3 to the proximal end of the multi-lumen tube 4 and to the proximal end of the sleeve 6 is illustrated in FIG. 5. The main body 31 of the connecting piece 3 has a tubular, cylindrical holding area 35 which forms an axial insertion opening 36. The likewise tubular compressed gas connector 34 protrudes diagonally from this holding area 35. The multi-lumen tube 4 has a lateral opening 45 which provides lateral access to the lumen 44. The multi-lumen tube 4 is first enclosed by the sleeve 6 in an area which begins distally from the lateral opening 45, so that the proximal end of the multi-lumen tube 4 protrudes from the sleeve 6. During assembly, the tube connectors 72, 73 of the adapter 7 are initially inserted into the lumina 42, 43 of the multi-lumen tube 4. Next, the proximal end of the multi-lumen tube 4, including the adapter 7, and the proximal end of the sleeve 6 are inserted, in the proximal direction, into the axial insertion opening 36. As described in greater detail in the following, the multi-lumen tube 4, the adapter 7, and the sleeve 6 are fixed in the holding area 35 by means of an adhesive.

The state of this area after assembly is illustrated in FIGS. 11 and 12.

As is evident from FIG. 11, in the inserted state, the lateral opening 45 of the multi-lumen tube 4 comes to rest in the extension of the bore hole of the compressed gas connector 34, and so the compressed gas can be fed from the compressed gas connector into the lumen 44. In a proximal area, the connecting piece 3 comprises an adhesive channel 37, which is open toward the lateral direction, in order to bring a UV-cured adhesive into the interior of the holding area 35. There, the adhesive fixes the proximal end of the multi-lumen tube 4 and the adapter 7. The quantity of adhesive is adjusted in such a way that adhesive does not enter the area of the lateral opening 45, in order to not block the compressed gas supply. In order to enable the adhesive to be cured by the effect of UV light, the connecting piece 3 is made from a UV-transparent plastic. Additionally, adhesive is introduced in the area of the insertion opening 36 between the holding area 35 and the sleeve 6, in order to fix the sleeve 6 on the holding area 35. Overall, the interior of the holding area 35 is sealed in the distal and the proximal directions by the adhesive, such that compressed gas cannot escape during operation.

In FIG. 12, it is clear that the inlet/outlet connectors 74, 75 of the adapter 7 have been inserted into two openings in a proximal end wall of the holding area 35. Through these openings, the inlet/outlet connectors 74, 75 of the adapter 7 communicate, in the proximal direction, with the reservoir connectors 32, 33. Through the component channels 77, 78 in the interior of the adapter 7, the inlet/outlet connectors 74, 75 also communicate, in the distal direction, with the lumina 42, 43 of the multi-lumen tube 4. Overall, in this way, the components can pass from the reservoir connectors 32, 33 into the lumina 42, 43. It is also apparent from FIG. 12 that the connecting piece 3 has a non-return valve 38 between the reservoir connectors 32, 33 and the inlet/outlet connectors 74, 75, respectively, of the adapter 7. As a result, compressed air is prevented from inadvertently passing through the lumina 42, 43 to the reservoir connectors 32, 33 during a malfunction, e.g., during an occlusion of the spray head.

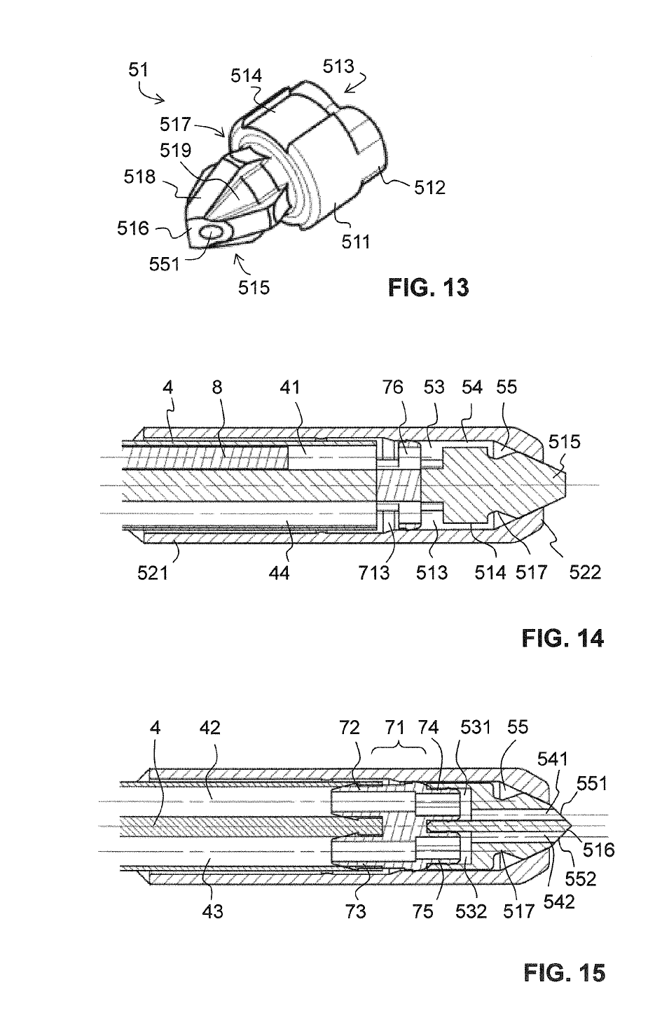

The design of the distal end of the applicator including the spray head 5 is illustrated in FIGS. 13-16.

The spray head insert 51 comprises a cylindrical main section 511. A spray head tip 515 protrudes from the main section 511 in the distal direction. Said spray head tip has, at its distal end, two slanted surfaces 516 which are disposed angled with respect to one another and in each of which a component outlet 551, 552 is located (see FIGS. 13 and 15). Formed in the spray head insert 51 are two component inlets 531, 532 which are open toward the proximal side of the spray head insert 51. The inlet/outlet connectors 74, 75 of the adapter 7 have been inserted into these component inlets 531, 532. Two parallel component channels 541, 542 extend in the distal direction through the main section 511 and the spray head tip 515, which component channels lead toward the outside at the component outlets 531, 532 (see FIG. 15). In this way, the components can travel from the lumina 42, 43 of the multi-lumen tube 4, through the adapter 7, and to the component outlets 531, 532 at the spray head tip.

A spacer 512 having a reduced cross-section adjoins the main section 511 in the proximal direction. Radially opposed, lateral recesses 513 are present in the area of the spacer 512. The recesses 513 are designed in such a way that the spacer 512, together with the main section 511, the spray head cap 52, and the adapter 7, delimits a hollow space 53 in the area of each of these recesses. After the compressed gas emerges from the compressed gas channel 76 of the adapter, it enters one of these hollow spaces 53 (see FIG. 14). In order to further conduct the compressed gas from there to the spray head tip, two radially opposite, flat recesses 514 are formed on the outer surface of the main section 511. These recesses 514, together with the spray head cap 52, form two channels 54 which distally adjoin the hollow spaces 52 in order to further conduct the compressed gas to the spray head tip 515.

Between the main section 511 and the spray head tip 515, the spray head insert 51 has a tapered area 517 having a reduced diameter. Together with the spray head cap 52, said tapered area forms an annular space 55 (see FIGS. 14-16). This annular space 55 accommodates gas passing through the channels 514.

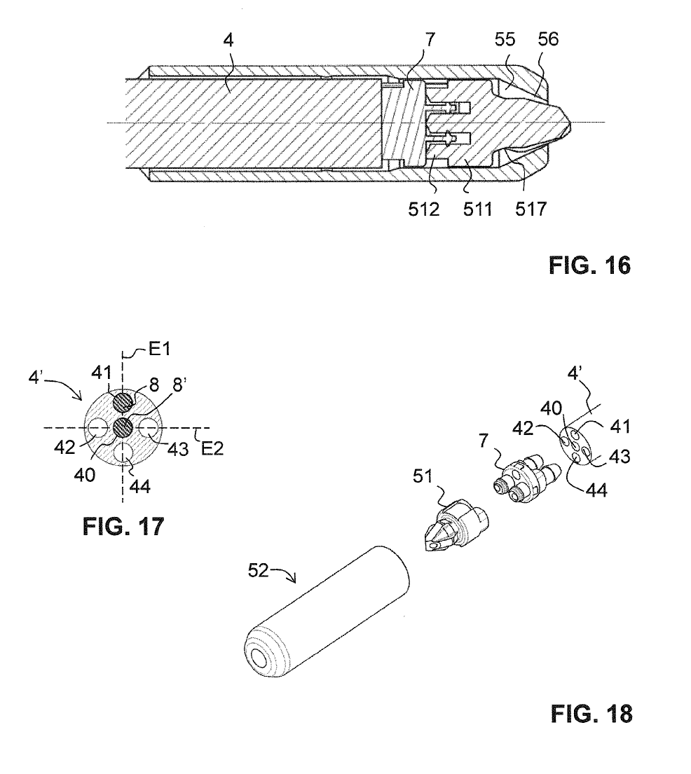

Between the tapered area 517 and the slanted surfaces 516, the spray head tip 515 comprises four axially extending webs 518 which rest, on the inside, against the end wall 522 of the spray head cap 52, which extends conically at a slant with respect to the longitudinal axis (see FIGS. 14 and 15). Four recesses 519 are formed between these webs 518. These recesses 519, together with the spray head cap 52, form four gas outlet channels 56 which lead, proceeding from the annular space 55, in the direction of the distal end of the spray head tip 515 (see FIG. 16). One of the webs 518 is disposed directly proximally adjacent to one of the slanted surfaces 516 in each case. As a result, the compressed gas, when it emerges from the gas outlet channels, does not flow directly over the component outlets 531, 532, but rather next to and past said outlets. This promotes good mixing of the components during the spraying and promotes a clean spray pattern.

As is the case with the adapter 7, the spray head insert 5 is also symmetrical with respect to an 180° rotation about the longitudinal direction and has mirror symmetry with respect to a horizontal plane and a vertical plane. The assembly of the applicator is additionally simplified as a result.

The guidance of the components and the gas at the spray head tip largely corresponds to the principles set forth in U.S. Pat. No. 8,534,575 B2. The content of this document is incorporated herein, in entirety, by reference. In particular, the spray head tip can be designed according to each of the variants disclosed in this document.

As is apparent in FIG. 14, a flexible wire 8 has been inserted into the first lumen 41. This wire is fixed, along its entire length, in the lumen 41 by means of an adhesive. Alternatively, the wire can also be fixed by way of being directly cast or extruded into the multi-lumen tube during the manufacture of the multi-lumen tube 4. As a result, the area of the multi-lumen tube 4 extending out of the sleeve 6 can be very exactly bent into a desired direction. Since the wire is fixed in the lumen, the bent area of the multi-lumen tube 4 retains its direction relative to the sleeve 6 also when the entire applicator is rotated about the longitudinal direction of the sleeve 6, even if a certain resistance (i.e., a certain torsional force) were to also act on the bent area. The stability in this regard is substantially greater than if the wire 8 were to be merely loosely placed into the multi-lumen tube 4. If necessary, it is also sufficient to only fix sections of the wire in the multi-lumen tube 4, e.g., at its ends. Given that the lumen 41, in which the wire is situated, extends decentrally offset from the longitudinal axis, the cross-section of the lumina 42, 43, which are provided for the guidance of the components, can be greater than is the case for a central arrangement of the wire-guiding lumen 41 (for a given diameter of the multi-lumen tube 4). As a result, it is simpler, on the one hand, to design the adapter 7, since the tube connectors 72, 73 must have a larger diameter than is the case for a central arrangement of the wire-guiding lumen 41. On the other hand, pressure losses over the length of the multi-lumen tube are minimized.

A second exemplary embodiment of a spray applicator is illustrated in FIGS. 16 and 17. Identical or identically acting parts are labeled using the same reference numbers as in the first exemplary embodiment. The second exemplary embodiment differs from the first exemplary embodiment merely by the design of the multi-lumen tube 4′ and by the number and arrangement of the wires 8, 8′ accommodated therein. The multi-lumen tube 4′ of the second exemplary embodiment comprises five instead of four lumina, wherein a central (i.e., extending on the central longitudinal axis of the tube) lumen 40 is additionally present, which lumen is enclosed by four decentral (i.e., extending offset with respect to the longitudinal axis) lumina 41-44. Whereas a single wire 8 is present in the first exemplary embodiment and this wire is accommodated in a decentral lumen 41, in the second exemplary embodiment, a further wire 8′ is additionally present, which is accommodated in the central lumen 40. The two wires, in turn, are fixed at least in sections, specifically preferably along their entire length, in the particular lumen. As a result, the area of the multi-lumen tube 4′ which extends out of the sleeve 6 has an anisotropic bending moment, i.e., the bending moment depends on the direction in which this area is intended to be bent. The area is stiffer for bending motions that take place in a plane E1 which extends through both wires 8, 8′ than for motions in a plane E2 perpendicular thereto.

The invention was explained above with reference to exemplary embodiments provided by way of example. Naturally, the invention is not limited to these exemplary embodiments, however, and a multiplicity of modifications is possible. For example, the spray head can be designed differently as is represented here. In particular, it can be provided that the spray head initially mixes the components in its interior before the thus mixed components reach the spray head tip. Naturally, the connecting piece can also be designed differently than the connecting piece 3 represented here by way of example, wherein the design can greatly depend on the type of discharge device from which the components are provided and how the compressed gas is fed. The sleeve 6 can also be longer or shorter than is represented here. If the sleeve 6 is flexible, it can also extend across the entire area from the connecting piece up to the spray head.

The above-described design of an applicator can also be utilized, instead of for laparoscopic applications, for other minimally invasive applications or for general endoscopic applications.

### LIST OF REFERENCE NUMBERS

1 Applicator

2 Discharge device

3 Connecting piece

31 Main body

32 First component connector

321 Securing sleeve

33 Second component connector

331 Securing sleeve

34 Compressed gas connector

35 Insertion area

36 Insertion opening

37 Adhesive channel

38 Non-return valve

4, 4′ Multi-lumen tube

40Central lumen

41 First lumen

42 Second lumen

43 Third lumen

44 Fourth lumen

45 Lateral opening

5 Spray head

51 Spray head insert

511 Main section

512 Spacer

513 Lateral recess

514 Recess

515 Spray head tip

516 Slanted surface

517 Area having a reduced diameter

518 Web

519 Recess

52 Spray head sleeve

521 Outer wall

522 End wall

523 Spray opening

53 Hollow space

54 Channel

55 Annular space

56 Gas outlet channel

531 First component inlet

532 Second component inlet

541 First component channel

542 Second component channel

551 First component outlet

552 Second component outlet

6 Sleeve/Concentric tube

7 Adapter

71 Base body

711 Main section

712 Spacer

713 Lateral recess

72 First tube connector

721 Retaining structure

73 Second tube connector

731 Retaining structure

74 First inlet/outlet connector

741 Sealing bead

75 Second inlet/outlet connector

751 Sealing bead

76 Compressed gas channel

L Longitudinal direction

1. An applicator for spraying at least two components in the interior of the body of a patient by using a compressed gas, comprising:
a connecting piece for connecting the applicator to reservoirs for the components and for feeding a compressed gas to the applicator;
a multi-lumen tube which defines a central longitudinal axis, with a proximal end, which is connected to the connecting piece, and with a distal end;
a spray head which is located at the distal end of the multi-lumen tube, and
at least one first flexible wire which is arranged in a first lumen of the multi-lumen tube; and
at least one adapter which is arranged at the proximal or distal end of the multi-lumen tube in order to connect the multi-lumen tube to the connecting piece or to the spray head,
wherein the adapter comprises two tube connectors which are introduced into a second and a third lumen of the multi-lumen tube, wherein the second and the third lumen extend off-center with respect to the longitudinal axis,
and wherein the first lumen, in which the first flexible wire is arranged, likewise extends off-center with respect to the longitudinal axis and offset from the second and the third lumen in the circumferential direction.

2. The applicator as claimed in claim 1, which comprises two adapters which are identically designed,
wherein one of the two adapters is arranged at the proximal end of the multi-lumen tube in order to connect the multi-lumen tube to the connecting piece, and
wherein the other adapter is arranged at the distal end of the multi-lumen tube in order to connect the multi-lumen tube to the spray head.

3. The applicator as claimed in claim 1, wherein the second and the third lumen extend diametrically opposite one another with respect to the longitudinal axis.

4. The applicator as claimed in claim 1, wherein the first flexible wire is fixed, at least in sections, in the multi-lumen tube.

5. The applicator as claimed in claim 1, which also comprises a second flexible wire which is arranged in a further lumen of the multi-lumen tube.

6. The applicator as claimed in claim 5, wherein the further lumen extends centrally along the longitudinal axis of the multi-lumen tube.

7. The applicator as claimed in claim 1, wherein the adapter comprises:
a base body which defines a first side and a second side with respect to the longitudinal direction, wherein the first tube connector and the second tube connector protrude from the base body on the first side,
a first inlet/outlet connector and a second inlet/outlet connector, wherein each of the inlet/outlet connectors protrudes from the base body, on the second side, and is designed for insertion into an opening of a further element,
a first component channel which extends through the base body and connects the first tube connector to the first inlet/outlet connector,
a second component channel which extends through the base body and connects the second tube connector to the second inlet/outlet connector, and
at least one compressed gas channel which connects the first side and the second side of the base body.

8. The applicator as claimed in claim 7, wherein the adapter connects the multi-lumen tube to the spray head,
wherein the spray head comprises a spray head insert and a spray head cap,
wherein the spray head insert comprises a first component inlet into which the first inlet/outlet connector of the adapter is inserted,
wherein the spray head insert comprises a second component inlet into which the second inlet/outlet connector of the adapter is inserted,
and wherein the spray head insert comprises at least one lateral recess which is designed in such a way that the spray head insert, together with the spray head cap and the adapter, delimits a hollow space in the area of the lateral recess, into which the compressed gas enters after having emerged from the compressed gas channel of the adapter before it emerges from the spray head.

9. The applicator as claimed in claim 8, wherein the spray head cap has an outer wall which radially surrounds the multi-lumen tube at its distal end, and wherein the spray head cap is bonded to the multi-lumen tube in the area of the outer wall by means of an adhesive.

10. The applicator as claimed in claim 9, wherein the adhesive is UV-curable, and wherein at least the outer wall of the spray head cap is permeable to UV light.

11. The applicator as claimed in claim 8,
wherein the spray head insert comprises a first component channel which is connected to the first component inlet and forms a first component outlet at the spray head tip,
wherein the spray head insert comprises a second component channel which is connected to the second component inlet and forms a second component outlet at the spray head tip, and
wherein multiple gas outlet channels are formed between the spray head insert and the spray head cap, which lead out of the spray head in an area adjacent to the component outlets.

12. The applicator as claimed in claim 7,
wherein the connecting piece comprises a first and a second reservoir connector for connection to the reservoirs, and a compressed gas connector for feeding the compressed gas,
wherein the adapter connects the reservoir connectors of the connecting piece to the multi-lumen tube,
wherein the connecting piece has a tubular holding area having an open distal end and a proximal end,
wherein the compressed gas connector leads into the interior of the tubular holding area;
wherein the multi-lumen tube is slid from the open distal end into the tubular holding area,
wherein the connecting piece has, at the proximal end of the holding area, a first component feed opening which communicates with the first reservoir connector and into which the first inlet/outlet connector of the adapter is inserted,
wherein the connecting piece has, at the proximal end of the holding area, a second component feed opening which communicates with the second reservoir connector and into which the second inlet/outlet connector of the adapter is inserted, and
wherein the multi-lumen tube has, in the area of the compressed gas connector, a lateral opening which connects the compressed gas connector to one of the lumina of the multi-lumen tube.

13. The applicator as claimed in claim 12,
wherein the multi-lumen tube and the adapter are fixed in the holding area by means of an adhesive, in an area which is located proximal to the lateral opening, and
wherein the multi-lumen tube is fixed in the holding area in a gas-tight manner in an area located distal to the lateral opening.

14. The applicator as claimed in claim 13, wherein the applicator comprises a sleeve which extends from the connecting piece in the distal direction and which radially surrounds at least a portion of the multi-lumen tube, and wherein the sleeve is fixed in the holding area by means of an adhesive, in an area located distal to the lateral opening.

15. The applicator as claimed in claim 13, wherein the connecting piece comprises an adhesive channel in order to feed the adhesive to the area located proximal to the lateral opening.

16. The applicator as claimed in claim 13, wherein the adhesive is UV-curable, and wherein the connecting piece is permeable to UV light at least in an area in which the adhesive is present.

17. An adapter for use in an applicator for spraying at least two components in the interior of the body of a patient by using a compressed gas, comprising:
a base body which defines a first side and a second side with respect to a longitudinal direction;
a first tube connector and a second tube connector, wherein each of the tube connectors protrudes from the base body, on the first side, and is designed for insertion into a lumen of a multi-lumen tube,
a first inlet/outlet connector and a second inlet/outlet connector, wherein each of the inlet/outlet connectors protrudes from the base body, on the second side, and is designed for insertion into an opening of a further element,
a first component channel which extends through the base body and connects the first tube connector to the first inlet/outlet connector,
a second component channel which extends through the base body and connects the second tube connector to the second inlet/outlet connector, and
at least one compressed gas channel which connects the first side and the second side of the base body.

18. The adapter as claimed in claim 17, wherein the base body has a lateral recess on the first side, and wherein the compressed gas channel terminates on the first side of the base body in the area of the lateral recess.

19. The adapter as claimed in claim 17,
wherein the adapter comprises two compressed gas channels,
wherein the base body has two lateral recesses which are disposed diametrically opposite one another, and
wherein both compressed gas channels terminate on the first side of the base body in the area of the lateral recesses.

20. The applicator as claimed in claim 4, wherein the first flexible wire is fixed in the multi-lumen tube at least at its ends.

21. The applicator as claimed in claim 4, wherein the first flexible wire is fixed in the multi-lumen tube essentially along its entire length.

22. The applicator as claimed in claim 13, wherein the multi-lumen tube is fixed in the holding area by means of an adhesive.
